(19) United States
(12) Patent Application Publication
(10) Pub. No.: US 2009/0186423 A1
(43) Pub. Date: Jul. 23, 2009

(54) Method for Characterization of a Recombinant Polyclonal Protein

(75) Inventors: Torben P. FRANDSEN, Frederiksberg (DK); Lone Kjaer RASMUSSEN, Skodsborg (DK); Anders ENGSTROM, Brommen (SE); Erland HOLMBERG, Sollentuna (SE); Pia PERSSON, Vallingby (SE)

Correspondence Address: STERNE, KESSLER, GOLDSTEIN & FOX P.L.L.C., 1100 NEW YORK AVENUE, N.W., WASHINGTON, DC, 20005, US

(73) Assignee: Symphogen A/S, Kgs. Lyngby (DK)

(21) Appl. No.: 12/275,095

(22) Filed: Nov. 20, 2008

Related U.S. Application Data
(60) Provisional application No. 60/996,647, filed on Nov. 28, 2007.
(60) Provisional application No. 60/996,574, filed on Nov. 26, 2007.

(30) Foreign Application Priority Data
Nov. 22, 2007 (DK) PA 2007 00765
Nov. 28, 2007 (DK) PA 2007 01687

(51) Int. Cl. G01N 33/563

(52) U.S. Cl. 436/512

(57) ABSTRACT

The present invention provides a characterization platform that can be used to assess the amount of different antibodies produced by a polyclonal cell line during production, as well as batch-to-batch consistency of the antibodies present in the polyclonal products. The structural characterization platform is based on removal of the heavy chains and separation of the light chains remaining via a chromatographic separation technique followed by mass spectrometry analysis on the intact light chain species.

This application claims the benefit of the filing date of U.S. Provisional Appl. No. 60/996,574, filed Nov. 26, 2007, U.S. Provisional Appl. No. 60/996,674, filed Nov. 28, 2007, Danish Appl. No. PA 2007 00765, filed Nov. 22, 2007, and Danish Appl. No. PA 2007 01687, filed Nov. 28, 2007, all of which are incorporated by reference in their entirety.

### BACKGROUND OF THE INVENTION

1. Field of the Invention

The present invention relates to a method for structural characterization of a population of different light chain species in a recombinant polyclonal antibody composition. The method is useful for both quantitative and qualitative analysis and can be used, for example, to analyse batch-to-batch consistency as well as to assess the compositional stability during a manufacturing run and to determine whether a given batch fulfils certain predefined release specifications.

2. Background Art

WO 2006/007853 discloses a procedure for characterizing a sample which comprises a recombinant polyclonal antibody. The method involves the digestion of the antibody chains to release a marker peptide which is unique for each specific protein species (so called ‘marker peptide’ method).

A prerequisite for industrial production of a recombinant polyclonal protein for prophylactic or therapeutic use is the maintenance of protein diversity during cultivation and downstream processing. Therefore, it is important to be able to monitor and measure the clonal diversity of a polyclonal cell line producing a polyclonal protein, as well as the relative representation of individual proteins in the polyclonal protein at any desired time point, and in any relevant sample, thus allowing for analysis of the stability of the expression system in a single run, as well as batch-to-batch variation of the final product.

Analysis of the batch-to-batch consistency in different drug substance batches produced from individual polyclonal working cell banks is needed to ensure that a particular batch is within pre-defined release specifications. Such an analysis would benefit from a method capable of determining the relative proportions of individual proteins in a polyclonal mixture of proteins.

The marker peptide method described in WO 2006/007853 provides an LC-MS (liquid chromatography-mass spectrometry) method for identification and characterization of unique hydrophobic variable region derived peptides generated by enzymatic digestion, which allows the identification of specific antibody species within a recombinant polyclonal antibody.

Adamczyk et al. (Rapid Communications in Mass Spectrometry 14, 49-51 (2000)) describe the analysis of a polyclonal antibody by purifying animal-derived (i.e. non-recombinant) polyclonal antibody, reducing the disulphide bonds between the light and heavy chains, and performing LC-MS on both heavy and light chains to provide a profile of the serum-derived polyclonal antibody.

Wan et al. (J. of Chromatography A 913, 437-446 (2001)) describe the use of LC-MS on a recombinant monoclonal antibody produced in CHO cells to quantify antibody glycoforms directly from the cell culture. Recombinant antibody samples from the cell culture are reduced and injected directly into an HPLC system, which is coupled to a mass spectrometer.

Further background to the invention is provided in WO 2006/007853.

### BRIEF SUMMARY OF THE INVENTION

The invention provides for a method for the characterisation of light chain species in a recombinant polyclonal antibody composition, said method comprising the steps of:

a) manufacturing and purifying a recombinant polyclonal antibody composition;

b) reducing the cysteine-bridges linking heavy and intact light chains;

c) separating heavy chains from intact light chains;

d) subjecting the intact light chains to at least one chromatographic analysis which separates proteins according to physico-chemical properties;

e) subjecting the separated intact light chains from step (d) to mass spectroscopy; and

f) analysing data obtained in step (e) to characterise the intact light chain species in the recombinant polyclonal antibody composition.

In order to decrease the complexity of the method and to improve the data set obtained from the isolated intact light chains, we have found it is necessary to separate the heavy chains from the light chains. We consider this is likely to be due to the high degree of heterogeneity in the physico-chemical properties of the heavy chains, which interfere with the characterization of the light chains. Furthermore, we have surprisingly discovered that when using intact light chains we obtain a more precise quantification of the composition of light chain antibodies in a recombinant polyclonal antibody. A further advantage in comparison to the marker peptide method is that the procedure is simplified with fewer steps, making it more robust and more convenient to use.

The intact light chain proteins to be characterized are typically derived from known genetic sequences, i.e. the sequences used to create the polyclonal antibody are known. Therefore, step (f) typically involves a comparison of the data obtained in step (e) with genetic data, such as the deduced molecular weight of each intact light chain as determined from the genetic sequence (or the other genetic analyses described herein), or step (f) involves a comparison of the data obtained in step (e) with data obtained from a molecular weight determination of isolated light chain species. The molecular weight of isolated light chain species can be obtained by expressing the antibody as a monoclonal antibody, separating light and heavy chains and determining the molecular weight of the light chain using mass spectrometry. A comparison of the data obtained in step (e) with data from a molecular weight determination will take post-translational modifications affecting the molecular weight into consideration.

While the present invention relates solely to analysis of the light chains, the end result may involve a determination of the amount and/or relative proportions of complete antibodies in the composition, because a 1:1 ratio always exists between a light chain and a heavy chain. It is possible to estimate the actual amount (on a weight basis) of each antibody species because the structure of the heavy chain associated with any given light chain is known in advance from its coding sequence. This can also be done by measuring the molecular weight of each isolated heavy chain using e.g. mass spectrometry in order to take post-translational modifications (in particular glycosylation) into account.

The invention also provides for a method for detecting variance between a population of intact light chains in two or more recombinant polyclonal antibody compositions, comprising performing the above method for the characterisation of light chain species in a recombinant polyclonal antibody composition, on each of the two or more recombinant polyclonal antibody compositions, and determining any variance between the populations of intact light chains in the two or more recombinant polyclonal antibody compositions.

### BRIEF DESCRIPTION OF THE DRAWINGS/FIGURES

FIG. 1. Typical chromatogram of SEC (size exclusion chromatography) of reduced and alkylated Sym001. HC=Heavy chain, LC=Light chain.

FIG. 2. Typical LC-MS chromatogram of Sym001 light chains. The total ion count (TIC) trace is shown at the top and the UV trace recorded at 214 nm is shown at the bottom.

FIG. 3. Typical UV chromatogram of Sym001 light chains with the retention times of the individual antibodies.

FIG. 4. TIC of Sym001 light chains (top) with the extracted ion chromatogram (XIC) of RhD159 (bottom).

FIG. 5. XIC of RhD159 (top) with the corresponding m/z spectrum.

FIG. 6. Enlargement of the m/z spectrum shown in FIG. 5 (top) with the corresponding XIC (bottom).

FIG. 7. Different amounts of Sym001 WS-1 LC injected, linearity of clones (n=3).

FIG. 8. Analysis of two different batches of Sym001 (n=3).

### DETAILED DESCRIPTION OF THE INVENTION

### Definitions

The term “anti-idiotype antibody” refers to a full-length antibody or fragment thereof (e.g. an Fv, scFv, Fab, Fab′ or F(ab)₂) which specifically binds to the variant part of an individual member of a polyclonal protein. Preferably, an anti-idiotype antibody of the present invention specifically binds to the variant part of an individual member of a polyclonal antibody or a polyclonal TcR. The anti-idiotype antibody specificity is preferably directed against the antigen-specific part of an individual member of a polyclonal antibody or a polyclonal T cell receptor, the so-called V-region. It may, however, also show specificity towards a defined sub-population of individual members, e.g. a specific VH gene family represented in the mixture.

The term “anti-idiotype peptide” refers to a specific peptide-ligand which is capable of associating specifically and thus identifying an individual protein member within a mixture of homologous proteins. Preferably, an anti-idiotype peptide of the present invention binds specifically to an individual member of a polyclonal antibody or a polyclonal TcR. The anti-idiotype peptides of the present invention are preferably directed against the antigen-specific part of the sequence of an individual antibody or an individual T cell receptor. An anti-idiotype peptide may, however, also show specificity towards a defined sub-population of individual members.

The term “clonal diversity” or “polyclonality” refers to the variability or diversity of a polyclonal protein, the nucleic acid sequences encoding it, or the polyclonal cell line producing it. The variability is characterized by differences in the amino acid sequences of individual members of the polyclonal protein or differences in nucleic acid sequences of the library of encoding sequences. For polyclonal cell lines, the clonal diversity may be assessed by the variability of nucleic acid sequences represented within the cell line, e.g. as single-site integrations into the genome of the individual cells. It may, however, also be assessed as the variability of amino acid sequences represented on the surface of the cells within the cell line.

The term “epitope” refers to the part of an antigenic molecule to which a T-cell receptor or an antibody will bind. An antigen or antigenic molecule will generally present several or even many epitopes simultaneously.

The term “antibody” describes a functional component of serum and is often referred to either as a collection of molecules (antibodies or immunoglobulins, fragments, etc.) or as one molecule (the antibody molecule or immunoglobulin molecule). An antibody molecule is capable of binding to or reacting with a specific antigenic determinant (the antigen or the antigenic epitope), which in turn may lead to induction of immunological effector mechanisms. An individual antibody molecule is usually regarded as monospecific, and a composition of antibody molecules may be monoclonal (i.e., consisting of identical antibody molecules) or polyclonal (i.e., consisting of different antibody molecules reacting with the same or different epitopes on the same antigen or on distinct, different antigens). The distinct and different antibody molecules constituting a polyclonal antibody may be termed “members”. Each antibody molecule has a unique structure that enables it to bind specifically to its corresponding antigen, and all natural antibody molecules have the same overall basic structure of two identical light chains and two identical heavy chains.

The term “immunoglobulin” is commonly used as a collective designation for the mixture of antibodies found in blood or serum. Hence a serum-derived polyclonal antibody is often termed immunoglobulin or gamma globulin. However, “immunoglobulin” may also be used to designate a mixture of antibodies derived from other sources, e.g. recombinant immunoglobulin.

The term “individual clone” as used herein denotes an isogenic population of cells expressing a particular protein, e.g. a monoclonal antibody. Such individual clones can for example be obtained by transfection of a host cell with a desired nucleic acid, and following selection for positive transfectants, a single clone may be expanded or a number of single clones may be pooled and expanded. A polyclonal cell line can be generated by mixing individual clones expressing different individual members of a polyclonal protein.

The terms “an individual member” or “a distinct member” denote a protein molecule of a protein composition comprising different, but homologous protein molecules, such as a polyclonal protein, where the individual protein molecule is homologous to the other molecules of the composition, but also contains one or more stretches of polypeptide sequence characterized by differences in the amino acid sequence between the individual members of the polyclonal protein, also termed a variable region.

For example, in a polyclonal antibody comprised of antibodies Ab1 to Ab50, all the proteins with the sequence of Ab1 will be considered as an individual member of the polyclonal antibody, and Ab1 may for example differ from Ab2 proteins in the CDR3 region. A sub-population of individual members can for example be constituted by the antibodies belonging to Ab1, Ab12 and Ab33.

The term “polyclonal antibody” describes a composition of different antibody molecules which is capable of binding to or reacting with several different specific antigenic determinants on the same or on different antigens. A polyclonal antibody can also be considered to be a “cocktail of monoclonal antibodies”. The variability of a polyclonal antibody is located in the so-called variable regions of the individual antibodies constituting the polyclonal antibody, in particular in the complementarity determining regions CDR1, CDR2 and CDR3. The polyclonal antibodies that may be characterized by the method of the invention may be of any origin, e.g. chimeric, humanized or fully human.

The terms “polyclonal manufacturing cell line”, “polyclonal cell line”, “polyclonal master cell bank (pMCB)”, and “polyclonal working cell bank (pWBC)” are used interchangeably and refer to a population of protein-expressing cells that are transfected with a library of variant nucleic acid sequences of interest. The individual cells that together constitute the recombinant polyclonal manufacturing cell line may carry only one copy of a distinct nucleic acid sequence of interest, encoding one member of the recombinant polyclonal protein of interest, with each copy preferably being integrated into the same site of the genome of each cell. Alternatively, each individual cell may carry multiple copies of a distinct nucleic acid sequence encoding a member of the recombinant polyclonal protein. Cells which can constitute such a manufacturing cell line can for example be bacteria, fungi, eukaryotic cells, such as yeast, insect cells or mammalian cells, especially immortal mammalian cell lines such as CHO cells, COS cells, BHK cells, myeloma cells (e.g., Sp2/0 cells, NS0), NIH 3T3, YB2/0 and immortalized human cells, such as HeLa cells, HEK 293 cells, or PER.C6.

As used herein, the term “polyclonal protein” refers to a protein composition comprising different, but homologous protein molecules, preferably selected from the immunoglobulin superfamily. Even more preferred are homologous protein molecules which are antibodies or T cell receptors (TcR), in particular antibodies. Thus, each protein molecule is homologous to the other molecules of the composition, but also contains at least one stretch of variable polypeptide sequence which is characterized by differences in the amino acid sequence between the individual members, also termed distinct variant members of the polyclonal protein. Known examples of such polyclonal proteins include antibodies, T cell receptors and B cell receptors. A polyclonal protein may consist of a defined subset of protein molecules, which has been defined by a common feature such as the shared binding activity towards a desired target, e.g. in the case of a polyclonal antibody against the desired target antigen. A recombinant polyclonal protein is generally composed of such a defined subset of molecules, where the sequence of each member is known. In contrast to a serum-derived immunoglobulin, a recombinant polyclonal protein will not normally contain a significant proportion of non-target-specific proteins.

The term “protein” refers to any chain of amino acids, regardless of length or post-translational modification. Proteins can exist as monomers or multimers, comprising two or more assembled polypeptide chains, fragments of proteins, polypeptides, oligopeptides, or peptides.

The term “unique marker peptides” describes a number of peptides originating from the variable region of the individual members of a polyclonal protein. The peptides are preferably generated by protease treatment or other means of protein fragmentation, and the peptides which can be unambiguously assigned to a single individual member of the polyclonal protein are termed unique marker peptides.

The term “recombinant polyclonal antibody” refers to a collection of antibodies manufactured using recombinant technology. In the context of the present invention, an antibody is considered recombinant if its coding sequence is known, i.e. also if it is expressed from a hybridoma or an immortalized B-cell. It will apparent, however, that the present invention is in particular directed to characterization of recombinant polyclonal antibody compositions where the antibodies are expressed using cell lines that are normally used for commercial production of recombinant antibodies, for example one of the human or other mammalian cell lines mentioned above. In the context of the present invention the term “recombinant polyclonal protein” includes a “recombinant polyclonal antibody”.

The recombinant polyclonal antibody according to the invention preferably comprises a population of at least two different antibodies, wherein at least the light chains differ.

All immunoglobulins independent of their specificity have a common structure with four polypeptide chains: two identical heavy chains, each potentially carrying covalently attached oligosaccharide groups depending on the expression conditions; and two identical non-glycosylated light chains. A disulphide bond joins a heavy chain and a light chain together. The heavy chains are also joined to each other by disulphide bonds. All four polypeptide chains contain constant and variable regions found at the carboxyl and amino terminal, respectively.

Immunoglobulins are divided into five major classes according to their heavy chain components: IgG, IgA, IgM, IgD, and IgE. There are two types of light chain, K (kappa) and λ (lambda). Individual molecules may contain kappa or lambda, but never both. IgG and IgA are further divided into subclasses that result from minor differences in the amino acid sequence within each class. In humans four IgG subclasses, IgG1, IgG2, IgG3, and IgG4 are found. In mouse four IgG subclasses are also found: IgG1, IgG2a, IgG2b, and IgG3. In humans, there are three IgA subclasses, IgA1, IgA2, and IgA3.

The term “intact light chain” refers to a recombinantly produced polypeptide which consists of both the variable and constant regions of a light chain polypeptide. The intact light chain is the product of expression of a light chain-encoding polynucleotide, taking into account post-translational modifications which may occur during production within an expression host and subsequent purification and/or processing.

An object of the present invention is to provide a platform for structural characterization to obtain information with respect to the presence or absence or relative proportion of individual antibodies in samples comprising a recombinant polyclonal antibody. The characterization platform can be used to assess different aspects during a process for production or purification of a recombinant polyclonal antibody or during long term storage of a recombinant polyclonal antibody composition.

Preferably, the characterization platform of the present invention is used for one of the following purposes i) to determine the relative representation of the individual members or some of the individual members in relation to each other within a single sample, ii) to assess the relative proportion of one or more individual members in different samples for determination of batch-to-batch consistency, and iii) to evaluate the actual proportion of one or more individual members. Optionally, this may be compared to the translated sequences in the expression vectors originally used to generate the polyclonal manufacturing cell line. The characterization platform can be used to monitor the clonal diversity of a polyclonal cell line and/or the representation of individual antibodies in a recombinant polyclonal antibody produced by the cell line. The characterization platform is particularly suited for both characterizing the compositional stability during individual production runs and for monitoring batch-to-batch consistency.

One embodiment of the present invention is a method for characterizing one or more samples which each comprise one or more recombinant polyclonal antibodies, where the polyclonal antibodies comprise multiple antibodies which differ by virtue of their variable regions, such that information is obtained with respect to the relative proportion or presence of the individual antibodies of the recombinant polyclonal antibody, said method comprising separating aliquots of isolated light chains from said samples by at least one chromatographic technique, and subsequently subjecting the isolated light chains to mass spectroscopy and optionally one or more genetic analyses of the protein-encoding sequences. The light chains may be either of the lambda or kappa isotype or a mixture of both lambda and kappa isotypes in the case of human antibodies, or other isotypes in the case of non-human antibodies.

It is an important feature of the present invention that the sequences coding for each cognate pair of heavy and light chains constituting the members of the polyclonal antibody are known. The information obtained from the analytical methods of the present invention relates solely to the light chains. By determining the amount of the different light chains in the polyclonal antibody, the amount of the complete antibodies can also be calculated, as the calculated molecular weight of each heavy chain is known from its coding sequence or determined experimentally using e.g. mass spectrometry.

In one preferred embodiment, the intact light chains comprise the entire light chain amino acid sequence, i.e. the light chain polypeptide produced by the manufacturing cell line, including post-translational processing which occurs during expression or secretion of the intact light chains.

In one embodiment, the intact light chains have an N-terminal amino acid residue other than glutamine, as it is conceivable that the N-terminal may be subjected to processing prior to the characterization. The C-terminal may also be subjected to processing.

In one embodiment, the chromatographic process is based on at least one physico-chemical property other than size.

In one embodiment, an individual chromatographic process is based on at least one physico-chemical property selected from the group consisting of net charge, hydrophobicity, isoelectric point, and affinity.

In one embodiment, an individual chromatographic process is based on net charge.

In one embodiment, the chromatographic process is performed as a multidimensional chromatography.

In one embodiment, the chromatographic process is or includes high resolution liquid chromatography.

In one embodiment, the polyclonal antibody composition is a cell culture fraction, such as a cell culture fraction comprising the cells of said culture. The cell culture fraction is typically a sample of the cell culture comprising cells representing each of the cell lines in the cell culture, so that the sample is representative of the larger cell culture.

In one embodiment, step (a) involves preparing a polyclonal antibody composition from one or more cell culture supernatants.

In one embodiment, the characterisation of antibody species in a recombinant polyclonal antibody composition involves the determination of the presence or absence of the light chain species in the recombinant polyclonal antibody composition.

In one embodiment, the characterisation of antibody species in a recombinant polyclonal antibody composition involves the determination of the relative proportion of the light chain species in the recombinant polyclonal antibody composition.

In one embodiment, the determination of the relative proportion of intact light chain species in a recombinant polyclonal antibody composition includes the analysis of one or more sentinel proteins present in said composition.

In one embodiment, step (f) comprises comparing the data obtained in step (e) with data obtained from at least one further analytic technique selected from the group consisting of a further protein characterization technique and a genetic technique.

In one embodiment, the at least one further analytic technique is a genetic analysis of the polynucleotides encoding the light chains, or polynucleotides obtained or derived from the manufacturing cell line.

In one embodiment, the genetic analysis is selected from RFLP, T-RFLP, microarray analysis, quantitative PCR and nucleic acid sequencing.

In one embodiment, a further characterization technique is a protein characterization technique selected from N-terminal sequencing and characterization of complex homologous protein mixtures with specific detector molecules such as anti-idiotype antibodies or anti-idiotype peptides.

In one embodiment, the at least one further analysis is performed prior to, during, or subsequent to steps a) to e).

The invention also provides for a method for detecting variance between a population of intact light chains in two or more recombinant polyclonal antibody compositions comprising performing the method for the characterization of light chain species as described herein on each of the two or more recombinant polyclonal antibody compositions, and determining any variance between the populations of intact light chains in the two or more recombinant polyclonal antibody compositions.

In one embodiment, the two or more recombinant polyclonal antibody compositions are obtained from a single polyclonal cell culture at different time points during the cultivation.

In one embodiment, the two or more recombinant polyclonal antibody compositions are obtained from different polyclonal cell cultures at a particular time point.

In one embodiment, the variance is detected by comparing the relative proportion of at least three, such as at least 5 or at least 10 intact light chains present in the two or more recombinant polyclonal antibody compositions.

In one embodiment, the variance is detected by comparing the relative proportion of at least two intact light chains present in the two or more recombinant polyclonal antibody compositions. Typically, the comparison is made with 50 or fewer intact light chains present in the two or more recombinant polyclonal antibody compositions, such as between 2-40, 2-30, 2-25, 2-20, 2-15, 2-10 or 2-5 intact light chains.

The recombinant polyclonal antibodies may be subject to optional additional characterization such as genetic and/or protein analyses. The genetic analyses refers to techniques such as deduction of the amino acid sequence and/or predicted mass from the genetic sequences encoding the intact light and heavy chains, restriction fragment length polymorphism (RFLP) analysis, terminal-RFLP (T-RFLP), microarray analysis, quantitative PCR such as real-time PCR, and nucleic acid sequencing. The protein characterization techniques refer to techniques generally used within the field of proteomics for characterizing unknown proteins, for example chromatographic analyses which separate proteins according to physico-chemical properties.

In addition to mass spectrometry, one or more of the following protein characterization techniques may be used—either, where appropriate, on the same sample, or more suitably on a parallel sample: analysis of proteolytic digestions of the homologous proteins, “bulk” N-terminal sequencing, and analysis using specific detector molecules for the homologous proteins. Genetic analyses of the clonal diversity of a polyclonal manufacturing cell line

In some embodiments of the present invention, the polyclonality in an expression system for producing a polyclonal protein is monitored by evaluating the quantity of cells encoding a particular member of the polyclonal protein in addition to the characterization methods of the present invention.

In addition to the protein characterization methods, one or more of the genetic analyses described herein may also be performed, including determination of the mRNA levels encoding individual members of the polyclonal protein. The genetic analysis may be monitored at the mRNA or genomic level using, for example, RFLP or T-RFLP analysis, oligonucleotide microarray analysis, quantitative PCR such as real-time PCR, and nucleic acid sequencing of the variable regions of the gene sequences obtained from (or used to create) the manufacturing cell line. Alternatively, the same techniques can be used to further qualitatively to demonstrate the (genetic) diversity of the polyclonal cell line. The nucleic acid sequences encoding the polyclonal protein can be monitored on samples obtained from a single polyclonal cell culture at different time points during the cultivation, thereby monitoring the relative proportions of the individual encoding sequences throughout the production run to assess its compositional stability. Alternatively, the nucleic acid sequences encoding the polyclonal protein can be monitored on samples obtained from different polyclonal cell cultures at a particular time point, thereby monitoring the relative proportions of the individual encoding sequences in different batches to assess batch-to-batch variation. Preferably, the sample used in the genetic analyses is a cell culture fraction enriched for the cells of the culture, e.g. by precipitation or centrifugation. In one embodiment, the genetic analysis can be performed on the manufacturing cell line(s) which produce the recombinant polyclonal antibody, whereas the chromatographic and mass spectroscopy analysis is performed on a polyclonal antibody sample obtained from the cell line. The sample for genetic analysis is generally obtained by harvesting a fraction of the cell culture at a desired time point, followed by removal of the medium, for example by centrifugation. Samples for comparison of batch-to-batch consistency are preferably obtained from cells at the limit for in vitro cell age for production.

In one embodiment, the genetic analysis may have been performed previously, such as sequencing of the genes which encode the individual light chains and which were used to create the manufacturing cell line(s). It is also envisaged that such genetic analysis may be performed simultaneously or after the protein characterization steps, such as the chromatographic and mass spectroscopy analyses.

Details of how to perform the genetic analysis techniques referred to herein are routine to the skilled person, and further guidance of how to perform RFLP/T-RFLP, oligonucletide microarray analysis, quantitative PCR and nucleic acid sequencing within the context of the invention is provided by WO 2006/007853.

### Separation of Heavy and Light Chains

One feature of the present invention is the separation of the heavy and light chains in a step preceding the mass spectrometry. This separation serves several purposes. First and foremost, it reduces the number of different protein sub-units in the sample. Secondly, antibody heavy chains, if manufactured in mammalian expression systems, are known to vary in their degree of glycosylation, so that each heavy chain is likely to give rise to several peaks in the chromatogram for the mass spectrometer. Thus, elimination of the heavy chains from the mass spectrometry step provides a better and more precise characterization of the antibodies.

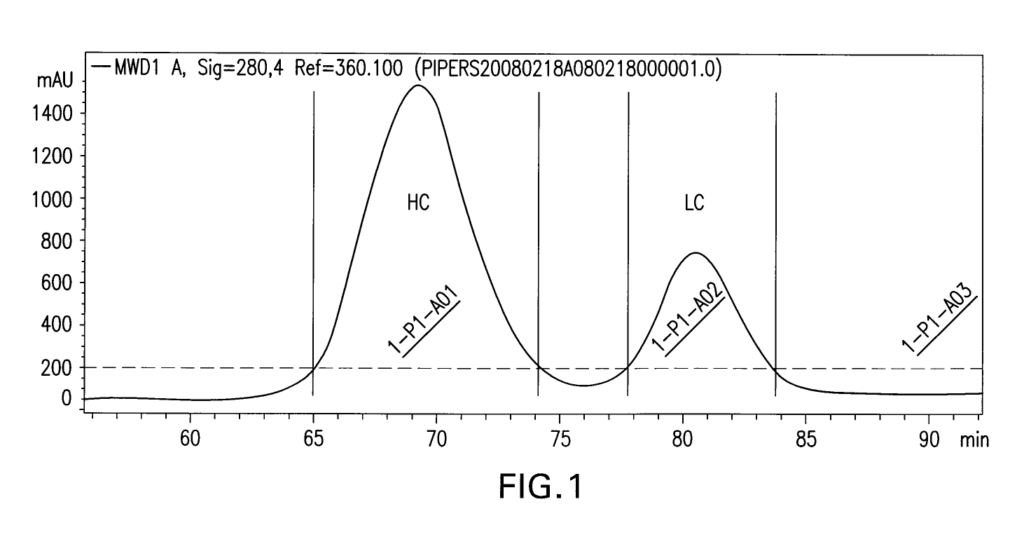

The separation of heavy and light chains can be carried out using size separation, such as gel filtration, which is sufficiently precise to separate the two groups of chains quantitatively (see FIG. 1). Other separation techniques may likewise be used, such as an affinity chromatography step, wherein heavy chains are retained while light chains are found in the flow-through.

### Mass Spectrometry

Mass spectrometric (MS) analysis is an essential tool for structural characterization of proteins. Mass spectrometric measurements are carried out in the gas phase on ionized analytes. By definition, a mass spectrometer consists of an ion source, a mass analyzer that measures the mass-to-charge ratio (m/z) of the ionized analytes, and a detector that registers the number of ions at each m/z value. Electrospray ionization (ESI) and matrix-assisted laser desorption/ionization (MALDI) are the two techniques most commonly used to volatize and ionize the proteins or peptides for MS analysis. ESI ionizes the analytes out of a solution and is therefore readily coupled to liquid-based (for example chromatographic and electrophoretic) separation tools. MALDI sublimates and ionizes the sample out of a dry, crystalline matrix via laser pulses. MALDI-MS is normally used to analyse relatively simple peptide mixtures, whereas integrated liquid-chromatographic ESI-MS systems (LC-MS) are preferred for the analysis of complex samples. The mass analyzer is central to the technology and its key parameters are sensitivity, resolution, mass accuracy and the ability to generate information-rich ion mass spectra from peptide fragments (MS/MS spectra). There are four basic types of mass analyzer currently used in proteomics research. These are the ion trap, time-of-flight (TOF), quadrupole and Fourier transform ion cyclotron (FT-MS) analysers. They are very different in design and performance, each with is own strength and weakness. These analysers can stand alone or, in some cases, be put together in tandem to take advantage of the strengths of each (for more details, see Aebersold & Mann, Nature 2003, 422:198-207).

In both MALDI- and ESI-MS, the relationship between the amount of analyte present and the measured signal intensity is complex and incompletely understood. Mass spectrometers are therefore inherently poor quantitative devices. Stable isotope protein labeling methods have been developed in the proteomic area to obtain quantitative MS data. These methods make use of the fact that pairs of chemically identical peptides of different stable isotope composition can be differentiated in a mass spectrometer due to their mass difference, and that the ratio of signal intensities for such peptide pairs accurately indicates the abundance ratio for the two peptides. Thus, relative abundance of their corresponding proteins in the original samples can be determined. Stable isotope tags can be introduced to proteins via i) metabolic labeling, ii) enzymatically, or iii) chemical reactions. Currently, chemical isotope-tagging of proteins or peptides is the most used method (for more details, see Aebersold & Mann, Nature 2003, 422:198-207). Increasing efforts have recently been directed to a label-free approach that relies on direct comparison of peptide peak areas between LC-MS runs. By varying the amount of a single protein or a few standard proteins, it has been shown that the intensities of peptide peak signals correspond nearly linearly to their concentrations in the sample, and that the ratios of peak areas between different LC-MS runs reliably reflect their relative quantities in the sample (Wang et al., J. Proteome Res. 2006, 5: 1214-1223).

### Chromatographic Separation Techniques

According to the present invention, the intact light chains are subjected to one or more chromatographic separation techniques (step d.).

Chromatographic separation of the individual members of the polyclonal protein may be based on differences in physico-chemical properties such as i) net charge (exemplified by ion-exchange chromatography (IEX)), ii) hydrophobicity (exemplified by reverse-phase chromatography (RP-HPLC), and hydrophobic interaction chromatography based on salt concentration (HIC)), iii) isoelectric point (pI value) (exemplified by chromatofocusing) or iv) affinity (exemplified by affinity chromatography using anti-idiotype peptides/antibodies, or protein-L chromatography for the separation of kappa and lambda antibody light chains). A fifth well known chromatographic technique is based on the physico-chemical property of size. However, this is not a particularly suitable technique for separation of homologous proteins such as antibody light chains, since all the light chains are of essentially the same size.

It is preferable that the chromatographic separation technique provides a sufficiently good separation of light chain species with identical or almost identical molecular weights, so that these can be subsequently distinguished in the mass spectrometer. The ability of the mass spectrometer to separate and distinguish between two light chain species with almost the same molecular weight decides which light chain species should be separated during the initial chromatographic step. Methods for achieving sufficient separation in the chromatographic separation technique lie within the capabilities of the person skilled in the art, who can adjust the buffer used, gradient, flow rate, pressure, column material, etc.

While in principle any chromatographic separation technique can be used, it is more convenient to use a method and a system that is compatible with the subsequent mass spectrometer, so that change of buffer can be avoided. The use of LC-MS is preferred since the two systems (liquid chromatography and mass spectrometry) are on-line, thus obviating the need for collection of fractions.

a) Ion-Exchange Chromatography

In some embodiments of the present invention, ion-exchange chromatography is used to separate individual light chain members of a recombinant polyclonal antibody or a sub-population of individual members of a polyclonal protein. The separation by ion-exchange chromatography is based on the net charge of the individual light chains in the composition to be separated. Depending on the pI-values of the light chains, and the pH values and salt concentrations of the chosen column buffer, the individual light chains can be separated, at least to some extent, using either anion or cation-exchange chromatography. For example, all the individual light chains will normally bind to a negatively charged cation-exchange media as long as the pH is well below the lowest pI-value of the individual light chains. The individual members of the bound light chains can subsequently be eluted from the column depending on the net charge of the individual proteins, typically using an increasing gradient of a salt (e.g. sodium chloride) or an increasing pH value. Several fractions will be obtained during the elution. A single fraction preferably contains an individual light chain member, but may also contain 2, 3, 4, 5, 6, 7, 8, 9, 10, 15, 20 or more distinct members. The general principles of cation and anion-exchange are well known in the art, and columns for ion-exchange chromatography are commercially available.

b) Chromatofocusing

In further embodiments of the present invention, chromatofocusing is used to separate individual light chain members of a recombinant polyclonal antibody or a sub-population of individual light chain members of a polyclonal antibody. The separation by chromatofocusing is based on differences in the pI values of individual proteins and is performed using a column buffer with a pH value above the pI value of the light chains. A recombinant polyclonal protein where the individual members have relatively low pI values will bind to a positively charged weak anion-exchange media. The individual light chain members of the bound recombinant polyclonal protein can subsequently be eluted from the column depending on the pI values of the individual light chain members by generating a decreasing pH gradient within the column using a polybuffer designed to cover the pH range of the pI values of the individual members. Several fractions will be obtained during the elution. A single fraction preferably contains an individual light chain member of the polyclonal protein, but may also contain 2, 3, 4, 5, 6, 7, 8, 9, 10, 15, 20 or more distinct light chain members. The general principles of chromatofocusing anion-exchangers are well known in the art, and anion columns are commercially available. Chromatofocusing with cation-exchangers is also known in the art (Kang, X. and Frey, D. D., 2003. J. Chromatogr. 991, 117-128).

c) Hydrophobic Interaction Chromatography

In further embodiments of the present invention, hydrophobic interaction chromatography is used to separate individual light chain members of a recombinant polyclonal antibody or a sub-population of individual light chain members of a polyclonal antibody. The separation by hydrophobic interaction chromatography is based on differences in hydrophobicity of the individual proteins in the composition to be separated. The recombinantly produced light chains are bound to a chromatography media modified with a hydrophobic ligand in a buffer that favors hydrophobic interactions. This is typically achieved in a buffer containing a low percentage of organic solvent (RP-HPLC) or in a buffer containing a fairly high concentration of a chosen salt (HIC). The individual light chain members are subsequently eluted from the column depending on the hydrophobicity of the individual light chain members, typically using an increasing gradient of organic solvent (RP-HPLC) or decreasing gradient of a chosen salt (HIC). Several fractions will be obtained during the elution. A single fraction preferably contains an individual light chain member of the polyclonal protein, but may also contain 2, 3, 4, 5, 6, 7, 8, 9, 10, 11, 12, 13, 14, 15, 16, 17, 18, 19, 20 or more distinct light chain members of the polyclonal protein. The general principles of hydrophobic interaction chromatography are well known in the art, and columns for RP-HPLC as well as HIC are commercially available. Mass spectrometers often have an HLPC unit linked directly to them, making the use of RP-HPLC as a prior separation step preferred.

d) Hydrophobic Charge Induction Chromatography

In further embodiments of the present invention, hydrophobic charge induction interaction chromatography (HCIC) is used to separate individual light chain members of a recombinant polyclonal antibody or a sub-population of individual light chain members of a polyclonal antibody. The separation by HCIC is based on differences in hydrophobicity of the individual proteins in the composition to be separated. Adsorption is based on mild hydrophobic interaction and is performed without the addition of salts. Desorption is based on charge repulsion achieved by altering the mobile phase pH. Optimal separation of the individual light chains, following adsorption to the HCIC resin, may be achieved by gradient optimization, e.g. by changing the pH and buffer salt in the mobile phase. A single fraction preferably contains an individual light chain, but may also contain 2, 3, 4, 5, 6, 7, 8, 9, 10, 11, 12, 13, 14, 15, 16, 17, 18, 19, 20 or more distinct light chains. The general principles of hydrophobic charge induction chromatography are well known in the art, and columns for HCIC are commercially available. An example of a commercially available HCIC resin is MEP HyperCel™ (PALL, East Hills, N.Y., USA). The MEP HyperCel™ sorbent is a high capacity, highly selective chromatography material specially designed for the capture and purification of monoclonal and polyclonal antibodies.

e) Affinity Chromatography

In further embodiments of the present invention, affinity chromatography is used to separate individual light chain members of a polyclonal antibody or a sub-population of individual light chain members of a polyclonal antibody. The separation by affinity chromatography is based on differences in affinity towards a specific detector molecule, ligand or protein. The detector molecule, ligand or protein, or a plurality of these (these different options are just termed ligand in the following), is immobilized on a chromatographic medium and the light chains are applied to the affinity column under conditions that favor interaction between the individual members and the immobilized ligand. Proteins showing no affinity towards the immobilized ligand are collected in the column flow-through, and proteins showing affinity towards the immobilized ligand are subsequently eluted from the column under conditions that counteract binding (e.g. low pH, high salt concentration or high ligand concentration). Several fractions can be obtained during the elution. A single fraction preferably contains an individual light chain member of the polyclonal antibody, but may also contain 2, 3, 4, 5, 6, 7, 8, 9, 10, 11, 12, 13, 14, 15, 16, 17, 18, 19, 20 or more distinct light chain members of the polyclonal antibody. The ligands which can be used to characterize a recombinant polyclonal protein are, for example, target-antigens, anti-idiotype molecules, or protein L for the separation of antibodies with kappa or lambda light chains.

Affinity chromatography with anti-idiotype molecules (e.g. anti-idiotype peptides or anti-idiotype antibodies) which specifically bind to individual members of a polyclonal protein or a sub-population of such individual members can be performed to obtain information with respect to the relative proportion of selected members of the recombinant polyclonal protein (also termed sentinel proteins), or a sub-population of individual members. Ideally, each individual anti-idiotype molecule only binds specifically to one individual member, but not to other members of the recombinant polyclonal protein, although an anti-idiotype molecule which binds a defined sub-set of members can also be used in the present invention. Preferably, anti-idiotype molecules are generated towards all the individual members, such that the complete polyclonal composition can be characterized. Where the recombinant polyclonal protein is a polyclonal antibody, the anti-idiotype molecules are directed against the antigen-specific part of the sequence of an antibody. The anti-idiotype molecules can be immobilized to the chromatographic medium individually, such that one column contains one anti-idiotype molecule, whereby information about a particular protein member or sub-population of proteins is obtained. The flow-through can then be applied to a second column with a second immobilized anti-idiotype molecule, and so forth. Alternatively, several different anti-idiotype molecules are immobilized on the same chromatographic medium applied to the same column. Elution is then performed under conditions that allow for the individual proteins to be eluted in different fractions, e.g. by adding increasing amounts of free idiotype molecules to the column, or using a pH or salt gradient. With this approach, it will be possible to obtain information on the proportions of several members of the polyclonal protein with a one dimensional analysis.

A polyclonal antibody may be composed of individual members which either contain a kappa light chain or a lambda light chain. In such a polyclonal antibody, the antibodies with a lambda light chain may be separated from the antibodies with a kappa light chain by using the lack of affinity towards Protein L for lambda light chain antibodies. Thus, a subset of antibody members containing the lambda light chain can be separated from a subset of antibody members containing the kappa light chain using Protein L affinity chromatography. The kappa and lambda antibody subsets can subsequently be characterized further using the characterization method of the invention.

### Multidimensional Chromatography

In general, one separation process is sufficient to obtain a good resolution of the light chains in the mass spectrometry step. Of course, this does not exclude the use of additional separation processes, which are described very briefly below.

Depending on the complexity of the variant homologous proteins in the sample to be analyzed, e.g. a recombinant polyclonal protein, it may be desirable to combine two or more of the chromatographic techniques described above in (a) to (e) in a two-dimensional, three-dimensional or multidimensional format. It is preferred to use liquid chromatography in all the dimensions instead of two-dimensional gel electrophoresis. However, this does not exclude the use of gel electrophoresis or precipitation techniques in one or more dimensions for the characterization of a recombinant polyclonal protein.

Generally, it is advantageous to use chromatographic techniques based on different physico-chemical properties in the different dimensions in a multidimensional chromatography, e.g. separation by charge in the first dimension, separation by hydrophobicity in the second dimension and affinity in the third dimension. However, some chromatographic techniques can provide additional separation when used in a subsequent dimension, even if they exploit similar physico-chemical properties of the protein. For example, additional separation can be obtained when chromatofocusing is followed by ion-exchange chromatography or affinity chromatography with different ligands which succeed each other.

As an alternative to multidimensional LC techniques, immunoprecipitation combined with a suitable electrophoresis technique, such as gel electrophoresis or capillary electrophoresis, and subsequent quantification of the antigens can be used to characterize a recombinant polyclonal protein. This technique will be particularly useful to characterize a recombinant polyclonal antibody targeted against complex antigens. A recombinant polyclonal antibody targeted against e.g. a complex virus antigen can be immunoprecipitated using a labeled antigen mixture and protein A beads. The antigens can subsequently be separated using isoelectric focusing or 2D PAGE followed by quantification of the individual antigens, reflecting the amount of antibodies in a recombinant polyclonal antibody targeted against the specific antigens.

### Elimination of N-Terminal Charge Heterogeneity in Recombinant Proteins

In the protein characterization techniques described in the above, heterogeneity of the individual protein in a pool of homologous proteins may complicate the characterization, since a single protein may result in several peaks in for example an IEX profile. Heterogeneity is a common phenomenon in antibodies and other recombinant proteins, and is due to enzymatic or non-enzymatic post translational modifications.

These modifications may cause size or charge heterogeneity. Common post-translational modifications include N-glycosylation (heavy chain only), methionine oxidation, proteolytic fragmentation, and deamidation. Heterogeneity can also originate from modifications at the genetic level, such as mutations introduced during transfection (Harris, J. R, et al. 1993. Biotechnology 11, 1293-7) and crossover events between variable genes of heavy and light chains during transcription (Wan, M. et al. 1999. Biotechnol Bioeng. 62, 485-8). These modifications are epigenetic and thus not predictable from the genetic structure of the construct alone.

Some of these post-translational modifications which may result in heterogeneity may be dealt with prior to characterization. Such modifications to facilitate characterization, without deletion of significant parts of the mature protein produced by the polyclonal manufacturing cell line(s), are in the context of the present invention considered to retain the intact light chain—i.e. the intact light chain may be modified, such as by one or more of the following techniques. In one embodiment such a ‘modified’ intact light chain consists of at least 90%, such at least 91%, such at least 92%, such at least 93%, such at least 94%, such at least 95%, such at least 96%, such at least 97%, such at least 98%, such at least 99%, such as 100% of the amino acid sequence of the mature intact light chain.

Charge variation arising from enzymatic removal of a C-terminal lysine can be solved by the use of specific carboxypeptidase inhibitors or by treating the antibody with carboxypeptidase to simplify the overall pattern (Perkins, M. et al. 2000. Pharm Res. 17, 1110-7).

Chemical degradation of proteins, such as deamidation, has been shown to be a significant problem during production and storage and to result in charge heterogeneity. Deamidation of Asn to Asp and formation of isoAsp (isoaspartyl peptide bonds) takes place under mild conditions (Aswad, D. W. et al. 2000. J Pharm Biomed Anal. 21, 1129-36). These rearrangements occur most readily at Asn-Gly, Asn-Ser, and Asp-Gly sequences, where the local polypeptide chain flexibility is high.

Charge heterogeneity may also result from N-terminal blockage by pyroglutamic acid (PyroGlu) resulting from cyclization of N-terminal glutamine residues (deamidation). Such post-translational modifications have been described for IgG as well as other proteins. Partially cyclization of the N-terminal of an antibody will result in charge heterogeneity, giving a complex IEX pattern. This problem cannot be solved by the use of the enzyme pyroglutamate aminopeptidase, first of all because the deblocking has to be performed on reduced and alkylated antibodies in order to obtain high yields of the deblocked antibodies (Mozdzanowski, J. et al. 1998, Anal. Biochem. 260, 183-7), which is not compatible with a subsequent IEX analysis, and second because it will not be possible to obtain a 100% cleavage for all the antibodies.

A further aspect of the present invention therefore relates to the elimination of charge heterogeneity caused by cyclization of N-terminal glutamine residues. The formation of N-terminal PyroGlu residues is eliminated by ensuring that no polypeptide chain contains an N-terminal glutamine, e.g. by changing said N-terminal glutamine residue to another amino acid residue. For antibodies, Gln residues at the N-terminal of the light chain may be exchanged. This is done by site-directed mutagenesis of nucleic acid sequences which encode polypeptides with an N-terminal glutamine. Preferably, the N-terminal glutamine residues are replaced by glutamic acid residues, since this is the uncharged derivative of glutamine. In a recombinant polyclonal protein, the individual sequences encoding the members may be changed and re-inserted into an expression vector to generate a new cell line expressing the changed protein. This cell line can then be included in the collection of cells producing the polyclonal protein.

### Further Characterization Techniques

In one embodiment of the present invention, the polyclonality of a pool of homologous proteins or the expression system for producing the homologous proteins is monitored by at least one further protein characterization technique. Such further protein characterization technique may be any technique that alone or in combination with other techniques is capable of providing information with respect to the presence and relative proportion of the individual members of a mixture of monoclonal proteins or a recombinant polyclonal protein in solution or on the surface of a cell present in a polyclonal cell line. Depending on the complexity of the recombinant polyclonal protein, one or more of the following techniques may be used: i) additional chromatographic separation techniques, ii) analysis of proteolytic digests of the polyclonal protein for identification of unique marker peptides representing individual members of the polyclonal protein, iii) “bulk” N-terminal sequencing, and iv) analysis using specific detector molecules, e.g. for characterization of sentinel protein members of the polyclonal protein. Suitably, the additional protein characterization techniques may be performed in parallel or even subsequent to steps d) and e).

In one embodiment, the further protein characterization technique is the analysis of proteolytic digests of the variable region of homologous proteins as referred to in WO 2006/007853. WO 2006/007853 also provides further instructions regarding the use of “bulk” N-terminal sequencing and characterization of complex homologous protein mixtures with specific detector molecules.

However, due to the advantages of the present method it is typical that no other protein characterization techniques are required in order to characterize the light chain species of the recombinant polyclonal antibody.

### Protein Sample

The polyclonal protein can for example be derived from a cell culture supernatant obtained from a polyclonal cell culture, e.g. in the form of a “raw” supernatant which only has been separated from cells e.g. by centrifugation, or supernatants which have been purified, e.g. by protein A affinity purification, immunoprecipitation or gel filtration. These pre-purification steps are, however, not a part of the characterization of the recombinant polyclonal protein since they do not provide any separation of the different homologous proteins in the composition. Preferably, the sample subjected to the characterization process of the present invention has been subjected to at least one purification step. Most preferred are samples which comprise at least 90% pure homologous proteins, such as at least 95% or more preferably 99% pure homologous proteins. Alternatively, the polyclonal antibody can be a mixture of separately manufactured and purified antibodies.

The different homologous proteins constituting the polyclonal protein can be monitored on samples obtained from a single polyclonal cell culture at different time points during the cultivation, thereby monitoring the relative proportions of the individual polyclonal protein members throughout the production run to assess its compositional stability. Alternatively, different homologous proteins constituting the polyclonal protein can be monitored on samples obtained from different polyclonal cell cultures at a particular time point, thereby monitoring the relative proportions of the individual encoding sequences in different batches to assess batch-to-batch consistency.

Complexity of a Mixture of Different Homologous Proteins to be Characterized

A sample to be characterized by the methods of the present invention comprises a defined subset of different homologous proteins having different variable region proteins, in particular different recombinant proteins. Typically, the individual members of a polyclonal protein have been defined by a common feature such as the shared binding activity towards a desired target, e.g. in the case of antibodies. Typically, a polyclonal protein composition to be analyzed by the characterization platform of the present invention will comprise at least 3, 4, 5, 10 or 20 distinct variant members (different homologous proteins). The polyclonal protein composition will thus typically comprise (at least) 3 different homologous proteins, such as (at least) 4, (at least) 5, (at least) 6, (at least) 7, (at least) 8, (at least) 9, (at least) 10, (at least) 11, (at least) 12, (at least) 13, (at least) 14, (at least) 15, (at least) 16, (at least) 17, (at least) 18, (at least) 19, (at least) 20, (at least) 21, (at least) 22, (at least) 23, (at least) 24 or (at least) 25 different homologous proteins, such as between 2 and 30 different homologous proteins, for example between 2 and 5, between 6 and 10, between 11 and 15, between 16 and 20, between 21 and 25 or between 26 and 30 different homologous proteins. In some cases, the polyclonal protein composition may comprise a greater number of distinct variant members, such as at least 50 or 100 different homologous proteins. Usually, no single variant member constitutes more than 75% of the total number of individual members in the polyclonal protein composition. Preferably, no individual member exceeds more that 50%, more preferably 25%, of the total number of individual members in the final polyclonal composition. In many cases, no individual member will exceed more than 10% of the total number of individual members in the final polyclonal composition.

In a preferred embodiment of the present invention, the sample comprising the different homologous proteins having different variable regions is a polyclonal antibody. The polyclonal antibody can be composed of one or more different antibody subclasses or isotypes, such as the human isotypes IgG1, IgG2, IgG3, IgG4, IgA1, and IgA2, or the murine isotypes IgG1, IgG2a, IgG2b, IgG3, and IgA.

The invention will be further described in the following non-limiting examples.

### EXAMPLES

### Example 1

### Preparation of a Recombinant Polyclonal Antibody

A recombinant polyclonal antibody composition containing 25 different individual anti-RhD antibodies was prepared according to Example 5 of WO 2006/007850. This polyclonal antibody composition is referred to below as “Sym001”.

### Example 2

### Isolation of Light Chains

According to the present invention, the identification of the individual antibodies is based upon the mass and retention time of the full-length light chain instead of only a peptide from the light chain. This feature simplifies the method (no enzyme is necessary), and thus improves the robustness of the method. The light chains (kappa) in Sym001, which are very similar to each other in sequence except for the CDR regions, do not contain post-translational modifications such as N-linked glycosylation, phosphorylation etc., and therefore could be expected to ionize more or less to same extent. Linearity of antibody response, recovery and reproducibility were evaluated. Two batches of Sym001 were also investigated to estimate the relative amounts of the individual antibodies in the different batches.

The sample was desalted by dialysis or using a PD10 column (GE Healthcare) against water, and A280 was monitored. The sample was then freeze-dried and reconstituted in 6 M Gua-HCl, 0.2 M Tris, pH 8.4 to a final concentration of 10 mg/ml and reduced and alkylated with DTT and iodoacetic acid, respectively.

The light chains of the sample were isolated on a Superose™ 12 10/300 GL size exclusion column (GE healthcare) on an Agilent 1100 HPLC system. The light chains were eluted with 6 M Gua-HCl, 50 mM NaP, pH 8.4 at a flow rate of 0.15 ml/min. Sample load: <1% of column volume.

A typical chromatogram of reduced and alkylated Sym001 is shown in FIG. 1.

### LC-MS

The light chain fraction was desalted by dialysis (Slide-A-Lyzer dialysis cassettes, 10000 MWCO, Pierce) against 0.1 M ammonium acetate, and A280 was measured. The analysis was performed on an Agilent 1100 HPLC connected on-line with an Agilent G1969A LC/MSD TOF mass spectrometer equipped with an ACE 3 C4-300, 100×2.1 mm, 3μ, column. The light chains were eluted with a gradient of acetonitrile in 0.04% trifluoroacetic acid with a flow rate of 0.4 ml/min operated at 60° C.

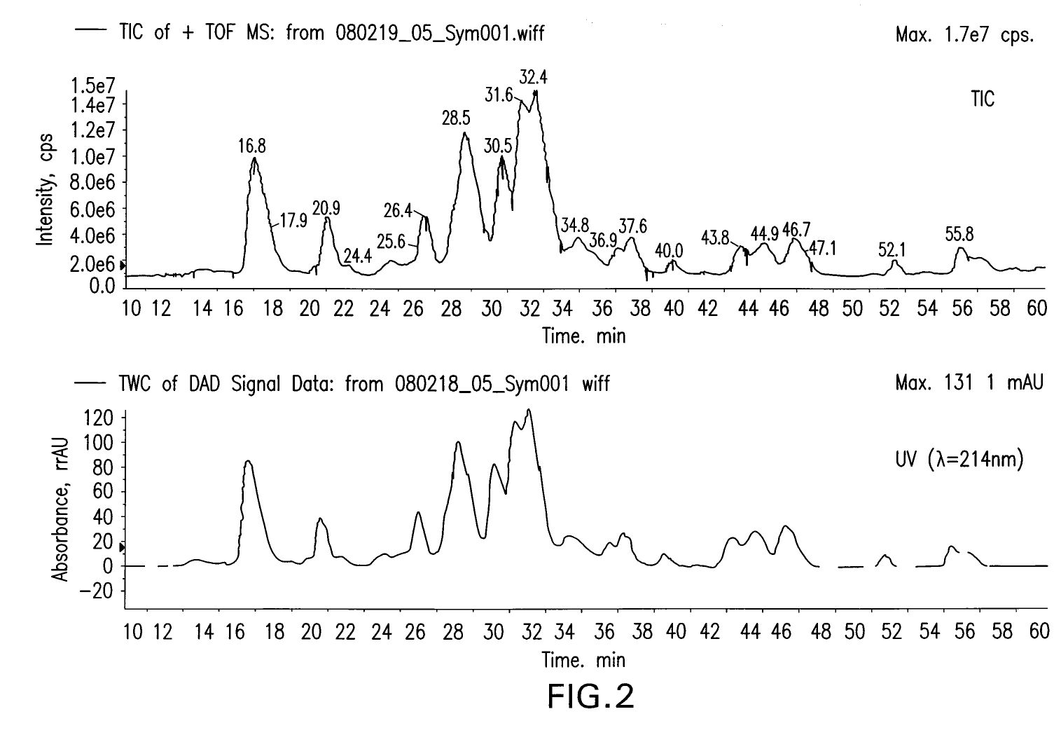

A representative chromatogram is shown in FIG. 2

### Evaluation—Identification and Quantitation

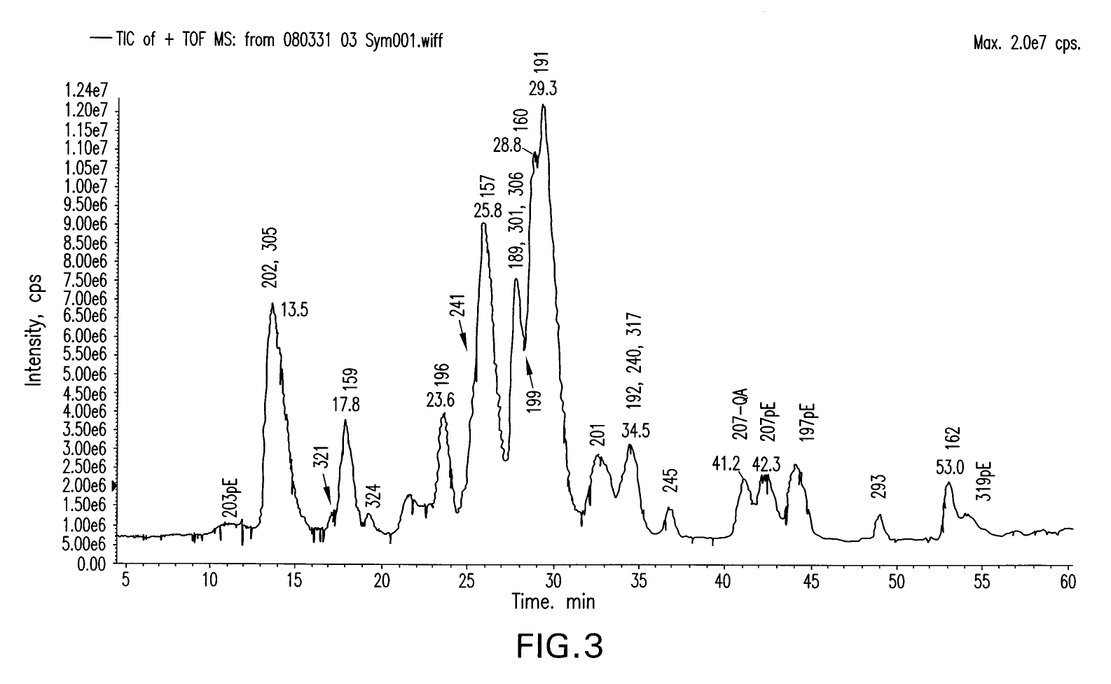

The identity of the individual light chains was established based on mass and retention time (FIG. 3).

Relative quantitation was achieved by plotting extracted ion chromatograms (XIC) of the most intense signals in the different light chain multiply charged envelopes and integrating their peak areas.

The software Analyst QS 1.1 (Agilent) was used for evaluation. Evaluation of one antibody is described below, RhD159 LC, with a mass of 23660.2.

### RhD159 LC

1) Identification of the m/z Peak with the Highest Intensity (Counts) in the m/z Spectrum

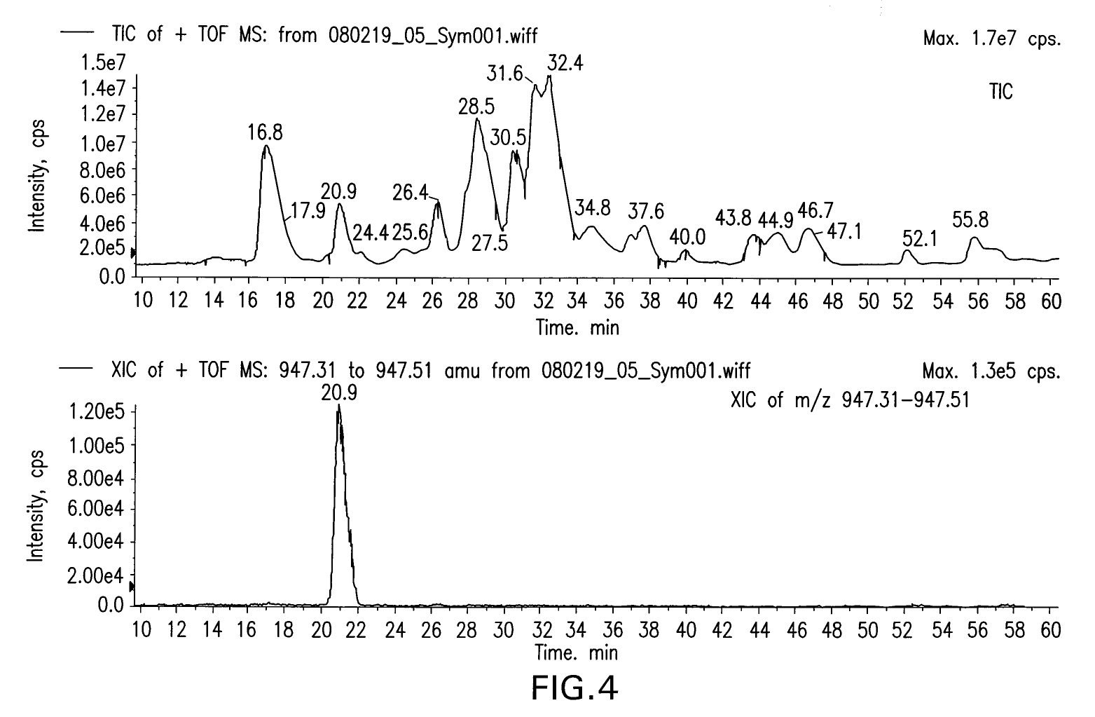

For antibody RhD159 (23660.2 Da), the theoretic m/z value of M+25H is 947.41. This is extracted from the TIC (total ion chromatogram) to elucidate a XIC (extracted ion chromatogram) shown in FIG. 4

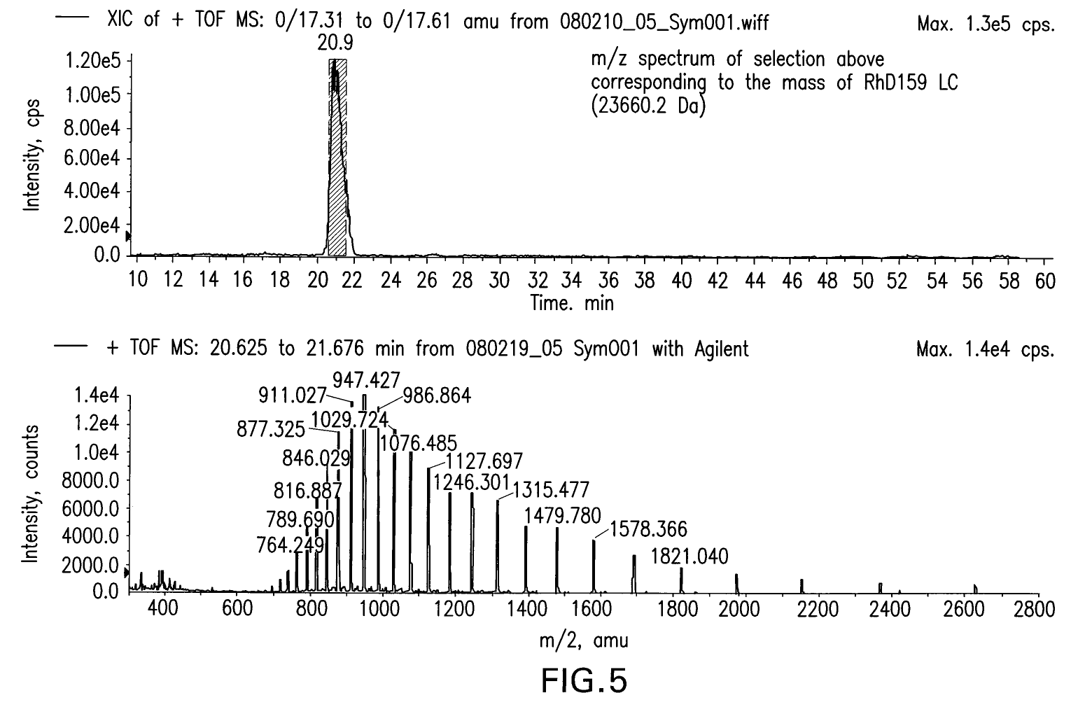

An m/z spectrum is extracted for the obtained peak time interval (FIG. 5).

The molecular ion with the highest intensity (counts) is 947.43 (M+25H).

2) Quantification (Determination of Peak Area) of the m/z Peak with the Highest Intensity (Counts) in the m/z Spectrum.

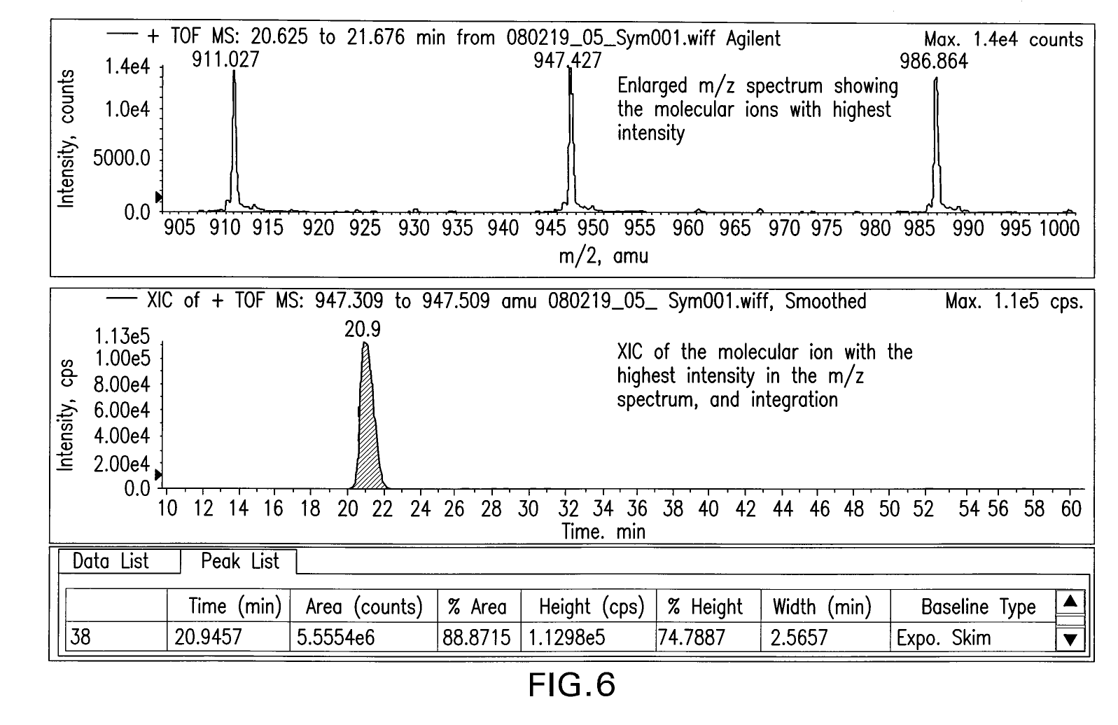

The molecular ion with the highest intensity (counts) is enlarged. It is extracted from the TIC using an extract ion tool which finds peak maximum and sets the m/z range automatically. The peak in the obtained XIC corresponding to RhD159 LC is integrated after smoothing (FIG. 6)

### Linearity

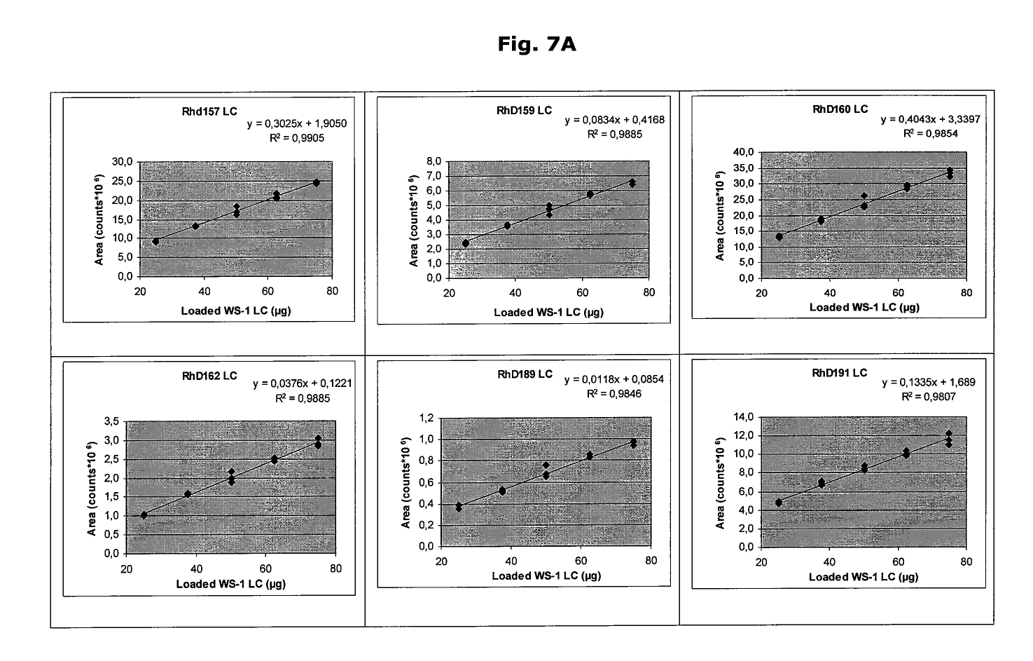

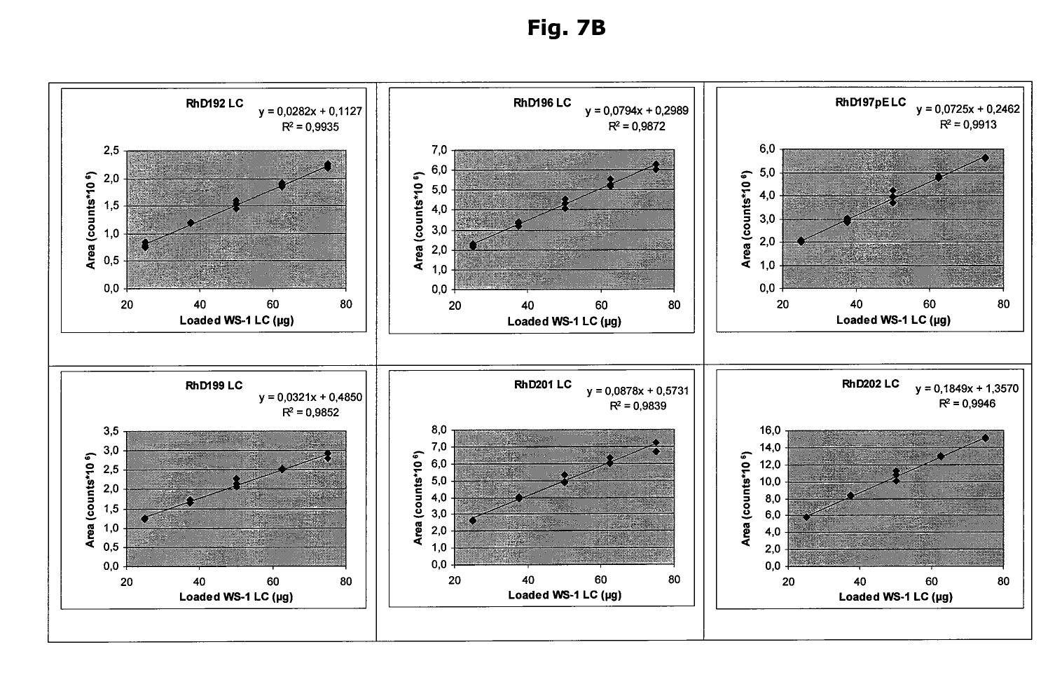

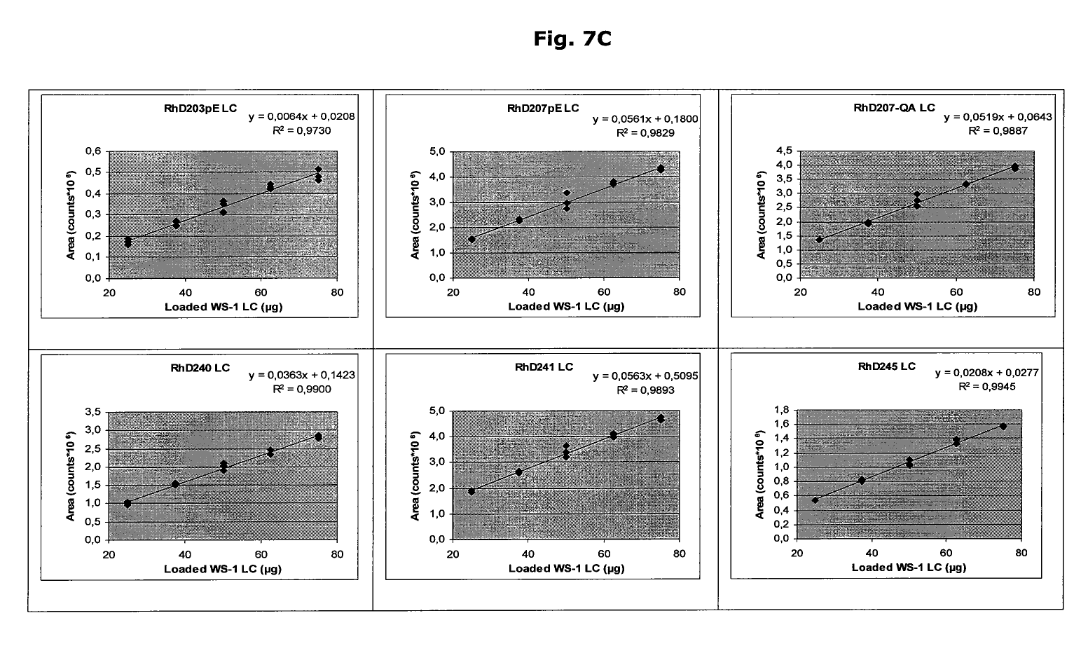

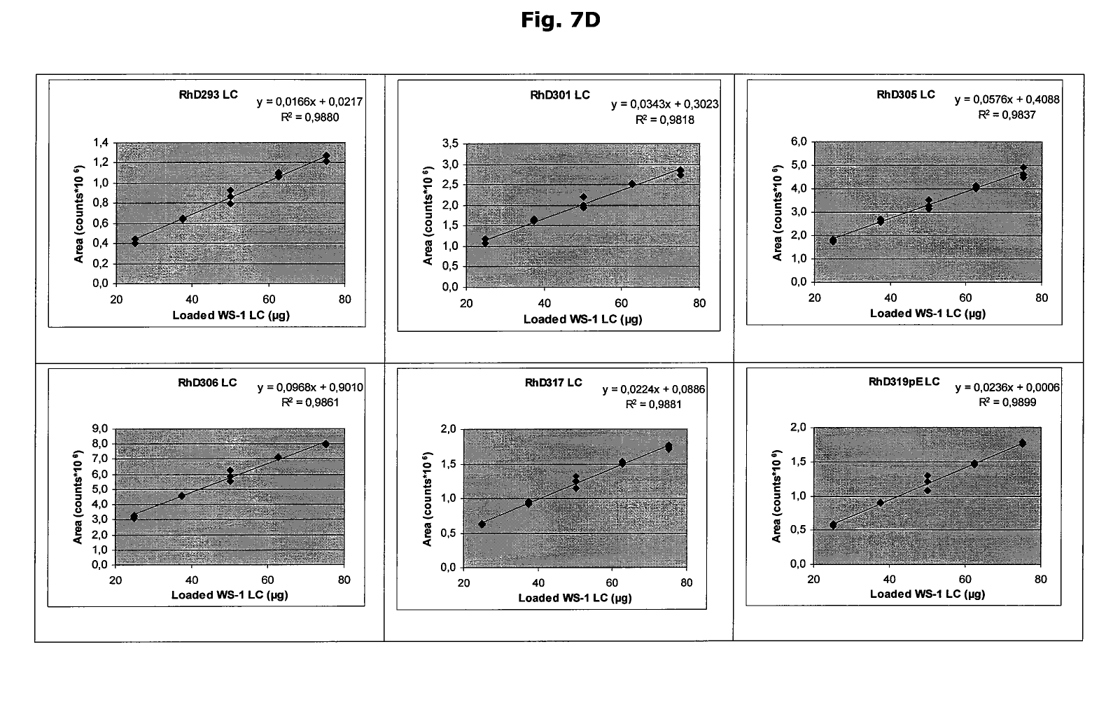

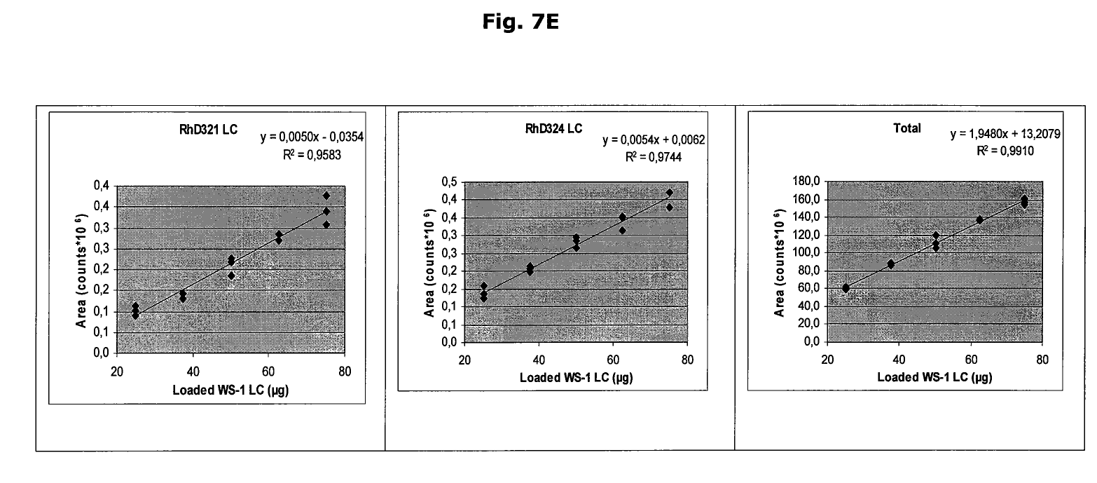

Linearity of antibody response was confirmed by injecting five levels (n=3) of Sym001 WS-1 LC (see FIG. 7).

### Recovery

Recovery was confirmed with spike-in experiments of the 25 individual antibodies constituting Sym001 as shown in Table 1. Each antibody light chain was analyzed individually at one or two levels, and spiked in Sym001 WS-1 LC at two levels.

TABLE 1
Recovery and linearity in spike-in experiments.

| | Recovery (%) | | Linearity (R²) | |
|---|---|---|---|---|
| Antibody LC | Level 1 | Level 2 | Ab alone | Ab in WS-1 LC |
| RhD157 | 88 | 101 | 0.9935 | 0.9943 |
| RhD159 | 121 | 112 | 1.0000 | 0.9980 |
| RhD160 | 98 | 101 | n.d | 0.9914 |
| RhD162 | 80 | 80 | n.d | 1.0000 |
| RhD189 | 108 | 107 | 0.9952 | 1.0000 |
| RhD191 (n = 3) | 81 | 74 | 0.9970 | 0.9909 |
| RhD192 | 120 | 121 | 0.9999 | 1.0000 |
| RhD196 | 104 | 101 | 0.9977 | 0.9998 |
| RhD197pE (n = 3) | 69 | 79 | 0.9996 | 0.9936 |
| RhD199 | 123 | 112 | 0.9994 | 0.9968 |
| RhD201 | 114 | 102 | n.d | 0.9926 |
| RhD202 | 98 | 87 | 0.9971 | 0.9943 |
| RhD203pE (n = 3) | 77 | 81 | 0.9998 | 0.9968 |
| RhD207 tot | 84 | 86 | 0.9997 | 0.9998 |
| RhD240 | 104 | 119 | 1.0000 | 0.9944 |
| RhD241 | 104 | 106 | 1.0000 | 0.9999 |
| RhD245 | 122 | 117 | 0.9971 | 0.9992 |
| RhD293 | 132 | 121 | 0.9956 | 0.9972 |
| RhD301 | 94 | 95 | n.d | 1.0000 |
| RhD305 | 71 | 78 | 0.9953 | 0.9974 |
| RhD306 | 85 | 79 | n.d | 0.9995 |
| RhD317 | 97 | 88 | 0.9860 | 0.9960 |
| RhD319pE (n = 3) | 78 | 82 | 0.9986 | 0.9981 |
| RhD321 | 95 | 104 | n.d. | 0.9965 |
| RhD324 (n = 3) | 134 | 128 | 0.9646 | 0.9994 |

n.d.: not determined

### Reproducibility—Relative Quantitation

Table 2 shows the results of the relative area calculated for each antibody light chain in Sym001 WS-1 analyzed on six different occasions. Two analysts performed six sample preparations using four preparations of reduction buffer and five preparations of the mobile phase using during SEC (size exclusion chromatography). Two SEC column lots were tested. The LC-MS part was performed with four preparations of mobile phase and two lots of the RPC (reversed phase chromatography) column. RSD (relative standard deviation) values were in the range of 1.1-8.4%.

TABLE 2
Relative area (%) of light chains in Sym001
WS-1 analyzed on six different occasions.

| Anti- | | | | | | | | | |
|---|---|---|---|---|---|---|---|---|---|
| body | Run | | | | | | Aver- | Std. | RSD |
| RhD | 1 | 2 | 3 | 4 | 5 | 6 | age | dev. | (%) |
| 157 | 15.4 | 15.4 | 15.5 | 15.5 | 15.1 | 15.2 | 15.4 | 0.16 | 1.1 |
| 159 | 4.1 | 4.2 | 4.1 | 4.4 | 4.2 | 4.4 | 4.2 | 0.13 | 3.1 |
| 160 | 21.7 | 22.1 | 22.0 | 21.2 | 20.5 | 20.9 | 21.4 | 0.63 | 2.9 |
| 162 | 1.8 | 1.6 | 1.6 | 1.8 | 1.9 | 1.7 | 1.7 | 0.13 | 7.4 |
| 189 | 0.6 | 0.6 | 0.6 | 0.6 | 0.6 | 0.6 | 0.6 | 0.02 | 3.5 |
| 191 | 7.3 | 7.0 | 7.1 | 7.3 | 6.9 | 7.2 | 7.1 | 0.17 | 2.3 |
| 192 | 1.3 | 1.5 | 1.5 | 1.5 | 1.5 | 1.5 | 1.5 | 0.06 | 4.3 |
| 196 | 3.8 | 3.8 | 3.7 | 3.9 | 4.0 | 3.8 | 3.8 | 0.10 | 2.6 |
| 197pE | 3.5 | 3.7 | 3.8 | 3.5 | 3.7 | 3.7 | 3.7 | 0.11 | 3.1 |
| 199 | 1.9 | 1.8 | 2.0 | 1.9 | 1.9 | 1.8 | 1.9 | 0.07 | 3.8 |
| 201 | 4.5 | 4.6 | 4.5 | 4.7 | 4.9 | 4.8 | 4.7 | 0.16 | 3.4 |
| 202 | 9.4 | 9.3 | 9.3 | 9.8 | 9.8 | 10.1 | 9.6 | 0.32 | 3.4 |
| 203pE | 0.3 | 0.3 | 0.3 | 0.3 | 0.3 | 0.3 | 0.3 | 0.02 | 6.0 |
| 207pE | 2.8 | 2.8 | 2.9 | 2.5 | 2.7 | 2.9 | 2.8 | 0.16 | 5.9 |
| 207-QA | 2.5 | 2.6 | 2.7 | 2.2 | 2.4 | 2.6 | 2.5 | 0.17 | 6.9 |
| 240 | 1.8 | 1.8 | 1.8 | 1.8 | 1.8 | 1.8 | 1.8 | 0.03 | 1.9 |
| 241 | 3.0 | 3.0 | 2.9 | 3.0 | 3.0 | 3.0 | 3.0 | 0.06 | 2.0 |
| 245 | 0.9 | 1.0 | 0.9 | 1.0 | 1.0 | 1.0 | 1.0 | 0.05 | 5.1 |
| 293 | 0.8 | 0.8 | 0.8 | 0.9 | 0.8 | 0.8 | 0.8 | 0.03 | 4.2 |
| 301 | 1.8 | 1.8 | 1.8 | 1.7 | 1.8 | 1.6 | 1.8 | 0.08 | 4.4 |
| 305 | 2.9 | 2.9 | 2.9 | 2.8 | 3.0 | 2.9 | 2.9 | 0.08 | 2.8 |
| 306 | 5.2 | 4.8 | 4.8 | 5.1 | 5.3 | 4.7 | 5.0 | 0.24 | 4.8 |
| 317 | 1.1 | 1.1 | 1.1 | 1.1 | 1.1 | 1.1 | 1.1 | 0.02 | 1.8 |
| 319pE | 1.1 | 1.1 | 1.0 | 1.1 | 1.1 | 1.1 | 1.1 | 0.03 | 2.6 |
| 321 | 0.2 | 0.2 | 0.2 | 0.2 | 0.2 | 0.2 | 0.2 | 0.02 | 8.4 |
| 324 | 0.2 | 0.3 | 0.3 | 0.2 | 0.2 | 0.3 | 0.3 | 0.02 | 6.6 |
| Sum | 100.0 | 100.0 | 100.0 | 100.0 | 100.0 | 100.0 | 100.0 | | |

pE indicates that the N-terminal Gln residue is cyclized to a pyroGlu. In the case of RhD207, the LC was found in two versions; as full-length and as a truncated form where the first two residues (QA) are missing due to processing by the signal peptidase.

### Analysis of Two Different Batches of Sym001

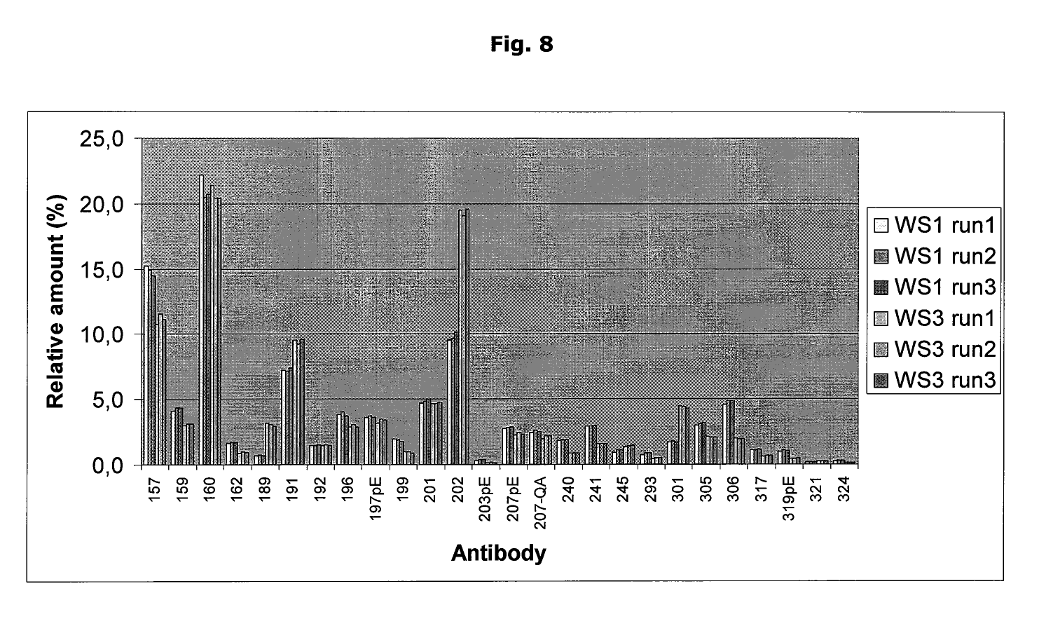

Two different batches were analyzed (n=3), and the results are shown in FIG. 8.

As seen in FIG. 8, the light chain LC-MS method of the invention is capable of detecting changes between two batches (see e.g. antibodies 157 and 202).

### CONCLUSION

We have developed an LC-MS based method by which we can identify and quantitate the 25 antibodies constituting Sym001:

An RP-HPLC method was developed to obtain resolution of light chains, especially those with close masses.

Masses corresponding to the light chain of all 25 antibodies were found in a Sym001 sample (Sym001 WS-1). For one antibody (RhD207), an additional truncated form was found.

The correct retention times have been verified for all 25 different light chains.

Linearity of antibody light chain response was confirmed by injecting different amounts of Sym001 WS-1 LC.

Recovery was confirmed with spike-in experiments of all 25 different light chains.

Reproducibility was tested with one sample, Sym001 WS-1 (n=6).

Two batches were analyzed (n=3), and it was shown that the light chain LC-MS method is capable of detecting changes between batches.

It will be appreciated by those of skill in the art to which this invention pertains that there are many conceivable variations in practicing the methods described herein. As such, there is no attempt made herein to provide all possible variations within the scope of this invention. All patent and non-patent documents cited herein are hereby incorporated by reference in their entirety for all purposes.

1. A method for the characterisation of light chain species in a recombinant polyclonal antibody composition, said method comprising the steps of:
a) manufacturing and purifying a recombinant polyclonal antibody composition;
b) reducing the cysteine-bridges linking heavy and intact light chains;
c) separating heavy chains from intact light chains;
d) subjecting the intact light chains to at least one chromatographic analysis which separates proteins according to physico-chemical properties;
e) subjecting the separated intact light chains from step (d) to mass spectroscopy; and
f) analysing data obtained in step (e) to characterise the intact light chain species in the recombinant polyclonal antibody composition.

2. The method according to claim 1, wherein the intact light chains comprise the entire light chain amino acid sequence.

3. The method according to claim 1 wherein the intact light chains have an N-terminal amino acid residue other than glutamine.

4. The method according to claim 1, wherein said chromatographic analysis is based on at least one physico-chemical property other than size.

5. The method according to claim 4, comprising an individual chromatographic analysis based on at least one physico-chemical property selected from the group consisting of net charge, hydrophobicity, isoelectric point, and affinity.

6. The method according to claim 5, wherein the individual chromatographic analysis is based on net charge.

7. The method according to claim 1, wherein said chromatographic analyses are performed as a multidimensional chromatography.

8. The method according to claim 1, wherein the chromatographic analysis is or includes high resolution liquid chromatography.

9. The method according to claim 1, wherein said polyclonal antibody composition is a cell culture fraction comprising the cells of said culture.

10. The method according to claim 1, wherein step (a) involves preparing a polyclonal antibody composition from one or more cell culture supernatants.

11. The method according to claim 1, wherein the characterisation of light chain species in the recombinant polyclonal antibody composition comprises determining the presence or absence of the light chain species in the recombinant polyclonal antibody composition.

12. The method according to claim 1, wherein the characterisation of light chain species in a recombinant polyclonal antibody composition comprises determining the relative proportion of the light chain species in the recombinant polyclonal antibody composition.

13. The method according to claim 1, wherein step (f) comprises comparing the data obtained in step (e) with data obtained from at least one further analytic technique selected from the group consisting of a further protein characterization technique and a genetic technique.

14. The method according to claim 13, wherein the at least one further analytic technique is a genetic analysis of polynucleotides encoding the light chains.

15. The method according to claim 13, wherein the genetic analysis is selected from RFLP, T-RFLP, microarray analysis, quantitative PCR and nucleic acid sequencing.

16. The method according to claim 13, wherein a further characterization technique is a protein characterization technique selected from N-terminal sequencing and characterization of complex homologous protein mixtures with specific detector molecules such as anti-idiotype antibodies or anti-idiotype peptides.

17. A method for detecting variance between a population of intact light chains in two or more recombinant polyclonal antibody compositions, comprising performing the method according to claim 1 on each of the two or more recombinant polyclonal antibody compositions and determining any variance between the populations of intact light chains in the two or more recombinant polyclonal antibody compositions.

18. The method according to claim 17, wherein the two or more recombinant polyclonal antibody compositions are obtained from a single polyclonal cell culture at different time points during the cultivation.

19. The method according to claim 17, wherein the two or more recombinant polyclonal antibody compositions are obtained from different polyclonal cell cultures at a particular time point.
